(19) United States
(12) Patent Application Publication
(10) Pub. No.: US 2006/0147911 A1
(43) Pub. Date: Jul. 6, 2006

(54) Method for characterizing primary tumors

(76) Inventors: Burkhard Hermann Brandt, Havixbeck (DE); Nicola Tidow, Munster (DE); Hartmut Schmidt, Georgsmarienhutte (DE); Axel Semjonow, Munster (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/511,527

(22) PCT Filed: Apr. 17, 2003
(86) PCT No.: PCT/EP03/04037

§ 371 Date: Apr. 21, 2005

(30) Foreign Application Priority Data
Apr. 17, 2002 (DE) 102 17 102.5

(51) Int. Cl. C12Q 1/68; C12P 19/34

(52) U.S. Cl. 435/6; 435/91.2

(57) ABSTRACT

The invention relates to a method for the detection and characterisation of primary tumours and separate areas of primary tumours, respectively. Clusters of tumour cells, extracted from sample material, are isolated and concentrated, followed by an analysis of genetic changes in these isolated cell clusters.

The innovative procedure presented here refers to a method for the characterisation of primary tumours and parts of tumours, respectively, using peripheral blood samples. Such methods are necessary for the evaluation of the degree of malignancy of a primary tumour, its invasiveness and its ability to form metastases.

Such methods are needed for all kinds of different tumour types, especially mamma-carcinoma, ovarian-, colon-, and stomach carcinoma, prostate and bladder carcinoma.

Prostate carcinoma (PCa) is on one of the most frequent causes of cancer-related death in the Western world. Prognostic criteria suggest three types of prostate cancer: 1) the small indolent carcinoma which during the life span of the patient does not grow to a clinically relevant or metastasising carcinoma; (2) the slow growing carcinoma which Is at first locally lymphatic and metastasises into the skeleton later on; (3) the carcinoma which metastasises early and spreads diffusely in the prostate and metastasises directly into the skeleton. Up to now there are only curative therapies available for those tumours which are detected at an early stage, i.e. when they are still restricted to the respective organ. Treatment methods are radical prostatectomy or radiotherapy. The optimal treatment method however is still subject to discussion. About 15% of those prostate carcinomas which were removed by radical prostatectomy were shown to have the same characteristics as asymptomatic autopsy findings and appear to be relatively benign, i.e. restricted to the organ and well differentiated with a small tumour volume. Although the natural development of these carcinomas is not fully understood as yet, it is assumed that they do possibly not require any treatment. However, about half of all prostate tissue samples taken after radical prostatectomy show a higher proportion of poorly differentiated, life-threatening carcinomas than would have been recognisable in pre-operative biopsies. This illustrates the poor predictability of the degree of malignancy. Hence the active treatment of clinically negligible carcinomas could possibly be the right decision. There Is no parameter to differentiate before onset of treatment between potentially life-threatening prostate carcinomas and those with a relatively benign or even asymptomatic progression.

The clinically established tumour marker PSA is not suitable for predicting the spread of metastases (Jhaveri et al. Urology November 1999; 54(5):884-90; Pound et al. JAMA May 5, 1999 281 (17):1591-7; Wolff et al. Eur Urol 1998; 33(4):375-81). Since these findings were published, a further serum parameter, i.e. free PSA, has become available. An improvement in the staging of patients however has not been achieved (Lin et al. Urology September 1998; 52(3):366-71).

The most frequently used method for the detection of circulating prostate carcinoma cells is the RT-PCR method for PSA mRNA. The first available studies demonstrate a higher diagnostic sensitivity and specificity for pre-operative stating using the PSA-RT-PCR in comparison to Image-giving methods, PSA in serum, and histological classification (Katz et al., Cancer 75, 1642-1648, 1995). Further studies demonstrate that the presence of PSA-mRNA in about ⅙ of patients with organ restricted tumours (pT2) and about a quarter of patients with extra capsular spread (pT3 tumours) is positive (Melchior et al. Clin. Cancer Res. February 1997; 3(2):249-56). However, not all patients with a positive PSA-mRNA result developed a progressive disease.

A further possible parameter for molecular staging is the mRNA of the prostate specific membrane antigen (PSMA or PSM) (Israeli et al., J.Urol. 153, 573-577). A high PSM expression has been found In PSA-negative, anaplastic tumours and bone metastases. The cDNA sequence of PSM is known so that studies were performed using RT-PCR for the demonstration of circulating PSM-positive cells in peripheral blood (Israeli et al., Cancer Res. 53, 227-230, 1993; Israeli et al. Cancer Res. 54. 1807-1811, 1994b; Israeli et al., J.Urol. 153, 573-577, 1995). Loric et al. confirmed by means of RT-PCR determination of PSM that a haematogenic spreading of prostate carcinoma cells already occurs in locally restricted tumours (pT2a and pT2b) (Loric et al., Clin.Chem. 41, 1698-1704, 1995). Some studies demonstrate a higher sensitivity of PSM-RT-PCR compared to PSA-RT-PCR in patients after prostate ectomy (Israeli et al., Cancer Res. 54, 1807-1811, 1994b). Other authors found the marker less sensitive in metastases-forming prostate carcinoma (Cama et al., J.Urol, 153, 1373, 1995) and also reported false positive results of PSM-RT-PCR in healthy controls (Lintula et al., J.Urol. 2, 155, 693A, 1996). Therefore, the clinical relevance of PSM-RT-PCR has to be clarified in further studies.

The determination of mRNA of human glandular kallikrein (hK2) could be a complimentary parameter for the determination of PSA mRNA. This protein has a prostate-specific expression pathway and a structural homology to PSA of 80%. In the study of Corey et al. only a third of PSA positive patients also had a positive result for hK2, whereas in 50% of those samples which were positive for hK2 the PSA-RT-PCR were negative (Corey at al., Urology 50, 184-188, 1997).

There are problems that arise in the illegitimate and the physiological expression of genes, but not in their tumour specific expression. Besides these, it can be stated from the biological rationale that the presence of circulating tumour cells as demonstrated by RT-PCR of mRNA of organ specific markers for the prostate, does not allow any conclusion as to the number of cells and their ability to metastasise. It is therefore necessary to search for further molecular markers.

All together there is a lack of predictive parameters to determine the type of carcinoma before surgical intervention. This is why the controversy as to the value of early diagnosis and the rating of surgical therapy of prostate carcinoma still remains. The question as to the ability of prostate carcinoma cells to metastasise also remains unanswered since 20% of patients with organ-restricted carcinomas and a negative bone scintiscan develop metastases in spite of a total removal of the primary tumour. On the other hand, 50% of patients with an operable prostate carcinoma will most likely not die of this cancer.

On the basis of this state of the art In technology the Innovation introduced here presents a method for the characterisation of primary tumours and parts of primary tumours, respectively. This method allows for a reliable staging and a reliable prognosis of tumours.

This task is being solved by a procedure according to claim 1 and the applicability according to claim 15. Advantageous Improvements of the innovative method in the dependent claims as well as the innovative method are permissible.

It is an advantage of the submitted innovative method that it is based on the analysis of short and simple repetitive sequences, i.e. of DNA, and in particular—but not exclusively—on the so called microsatellite DNA.

It is scientifically well acknowledged that there is a connection between the formation and spread of malignant tumours and an accumulation of multiple genetic changes, i.e. these changes affect genes for cell cycle control or for cell differentiation. Short polymorphic DNA sequences, at least one base in length, could be used as sensitive markers for these changes. One well-researched group of these polymorphic sequences are the so called microsatellites which consist of 10 to 60 repetitive sequences of 2 to 6 base pairs and have a length of <1 kb. This has already been well described In the literature: Loeb L. A., Cancer Res. 51: 3075-3079, 1991: Fearon E. R., Vogelstein B. Cell 61: 759-767, 1990: Peltomaki P. et al., Science 260: 810-812, 1993: Isaacs, W. B. Carter, B. S. Cancer Survival 11: 15-24, 1991; Kunimi, K. et al., Genomics, 11: 530-536, 1991; Suzuki, H.; Komiya, A.; Aida, S.; Akimoto, S.; Shiraishi, T.; Yatani, R.; Igarashi, T.; Shimazaki, J., Cancer Res., 6: 956-61, 1995,; Uchida, T. et al., Oncogene 10: 1019-1022, 1995; Berthon, P. et al., Br. J. Cancer 72: 946-51, 1995; Carter, B. S. et al., PNAS 87: 8751-5, 1990; Egawa, S. et al., Cancer Research 55: 2418-2421, 1995; MacGrogan, D. et al., Genes, Chromosomes and Cancer 10: 151-9, 1994; Macoska, J. A. et al., Cancer Research 54: 3824-3830, 1994; Bova, G. S. et al., Cancer Research 63: 3869-3873, 1993; Gao, X. et al., Cancer Research, 55: 1002 1005, 1995; Macoska J. A. et al., Cancer Research 55: 5390-5395, 1995; Suzuki H. et al. Genes, Chromosomes and Cancer, 13; 168-74, 1995; Trapman J. et al., Cancer Research, 54: 6061-6064, 1994; Vocke C. D. et al., Cancer Research, 56: 2411-2416, 1996; Cheng L. et al., J. Nad Cancer Inst Feb. 4, 1998 90(3):233-7; Takimoto Y. et al., Cancer Jan. 15, 2001; 91(2):362-70.

In the invented method presented here, alterations in such microsatellite DNA were tested and by demonstrating genetic changes, evidence, characterisation, quantification and a prognosis for tumours were achieved. Tumours can be differentiated in to those that are proliferative, non-proliferative or apoptopic. The degree of malignancy, the invasiveness in affecting other organs, and the formation of metastases can be determined with this innovative method by genotyping cells from cell clusters. In particular, for instance, Isolated tumour cells from blood samples can to allocated to separate areas of a multi-focal tumour, i.e. Its ability to clone can be determined. With this kind of grading an outcome prediction and a subtle classification of primary tumours is possible.

In particular, this is possible when cell clusters of tumour cells are isolated from either blood samples, fluid from nipple aspiration of the female breast, urine or tissue samples.

Particularly advantageous has been the analysis of those microsatellites which are described in claim 4. For some of these microsatellites, as also stated in claim 6, a multiplex PCR as been developed for the amplification of DNA. In particular, the choice of microsatellites and the primer for the multiplex PCR as stated In claim 8, led to the effect that the microsatellites of each multiplex PCR preparation were spread over as many chromosomes as possible. The amount of amplified fragments among the different microsatellites varied so much that a separation, for Instance by means of capillary electrophoresis, was possible without any problems.

The separation and evaluation of PCR can for Instance be carried out on an automated system such as the ABI Prism 310 Genetic Analyse™. Reproducible amplification patterns are possible in a concentration range of 100 ng down to 1 ng of prepared DNA. The examined genomic alterations of the microsatellites DNA refer on the one hand to the so called LOH value (loss of heterozygosity) and on the other hand to the RER value (replication error).

For the calculation of the LOH the formula published by Canzian et al. Cancer Res. Jul. 15, 1996; 56(14)3331-7 was used:
LOH score=peak area allele 2 tumour×peak area allele 1 normal tissue/peak area allele 1 tumour×peak area allele 2 normal tissue.

This formula is based on test results which were achieved with an analogous genetic analysis system. This calculation entails the ratio of peak areas of alleles in one sequence. In Table 1 the marker D13S153 is used to demonstrate that the quotients of peak areas with low variation coefficients can be determined. Therefore the multiplex PCR protocols of this Innovative method allow for a reproducible and sensitive determination of an LOH.

TABLE 1
Comparison of the quotients for alleles 1 and 2 in MCF-7-cells

| | | | Quotient |
|---|---|---|---|
| PCR-Nr. | D13S153 | D13S153 A2 | A1/A2 |
| 1 | 9227 | 11393 | 0.8 |
| 2 | 5593 | 6431 | 1.14 |
| 3 | 7663 | 8315 | 0.92 |
| 4 | 13123 | 12544 | 1.04 |
| 5 | 9674 | 10576 | 0.91 |
| 6 | 9538 | 9405 | 1.01 |
| 7 | 11847 | 11137 | 1.06 |
| 8 | 8240 | 7896 | 1.04 |
| 9 | 11125 | 12090 | 0.92 |
| 10 | 12197 | 11325 | 1.07 |
| Medium | | | 0.991 |
| Standard deviation | | | 0.10082438 |

The calculation of a replication error (RER) also takes into account the length of fragments defined as a factor which represents the crucial point of peak distribution.

The lower detection limit for multiplex PCR with three primer pairs was determined In DNA from cell lines SK-BR 3 and LNCaP as well as In patient DNA (comparison tumour DNA leucocyte DNA). A reproducible line pattern was achieved for all polymorphic markers up to a concentration of >1 ng DNA. This corresponds to a number of about 50 cells.

Preferably, the tumour cells to be tested, from e.g. a blood sample, should be isolated or concentrated by first adding epithelial cells by means of density gradient centrifugation, followed by immuno-magnetic isolation or concentration of cytokeratin-positive cell clusters and/or PSA-positive cell clusters. Hereby magnetic beads with the corresponding antibodies are used. The density gradient centrifugation is performed according to the method described by Brandt and Griwatz, Clin.Chem. 42, 11, 1996:1881-1882. This article in its entity is herewith Included in the present patent application. The Immuno-magnetic Isolation of cells Is performed according to the method described by Griwatz et al., J.Immunol.Meth., 183.1995:251-265. The following antibodies were used as primary antibodies;

Rabbit-mouse anti-PSA, mouse anti-cytokeratine-biotinylised, mouse anti-cFas. mouse anti M30, mouse anti-Mib1 and mouse anti-H1/H3 histon proteins and. Secondary antibodies: anti rabbit and anti mouse with Alexa 488 and Alexa 594 or FITC, Cy5, Cy3, RPE.

Crucial in the isolation method described here is that for the molecular staging only cell clusters are used which were isolated by the above mentioned method. It has proved to be particularly advantageous to use agents with hyper-osmolarity during the density gradient centrifugation. This causes the cells in the cell clusters to shrink so that during the following immuno-magnetic cell isolation the columns are not blocked by cell clusters. This leads to a vastly increased yield of tumour cells from the blood sample so that almost exclusively tumour cells are found on the microscopic slide.

It turned out that when using this method isolated cells were mainly found as cell clusters which are positive for PSA and cytokeratine. All patients with prostate carcinoma had such cell clusters whereas the controls were negative for such cell clusters. The size of the cell clusters ranged from 2 to 70 cells, whereby the number of dusters in 20 ml peripheral blood was between I and 5400. 90% of patients however had more than 100 cells (and hence exceeded the detection limit.

Based on the cell morphology and the nuclear staining, two classes of cell dusters can in principle be identified: In large numbers there were clusters consisting of dysmorphic cells. In some cases there were small, round, nucleus-containing cells enclosed in these cells. Further, 25 of the 74 examined patients with prostate carcinoma had clusters which consisted only of small, round and nucleus-containing cells of about 5-7 μm diameter. Most of the patients (about 60%) had less than 10 such cell clusters in 20 ml blood. In three cases however up to 200 of such cell clusters were detected.

Both groups of cells clusters differ in detection rates in apoptose markers cFas and M30 as well as the proliferation marker Mib-1 and H1/H3. Dysmorphic cell clusters were positive for marker cFas and M30 whereas the group of small, round, nucleus-containing cell clusters were negative.

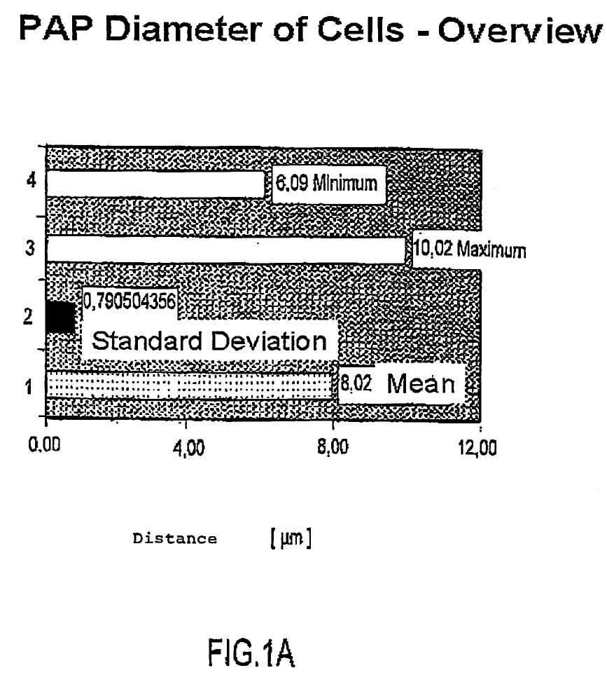

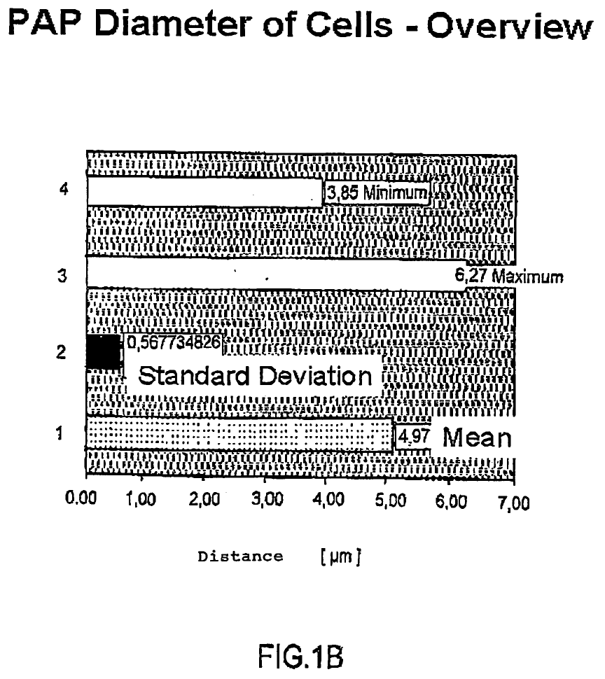

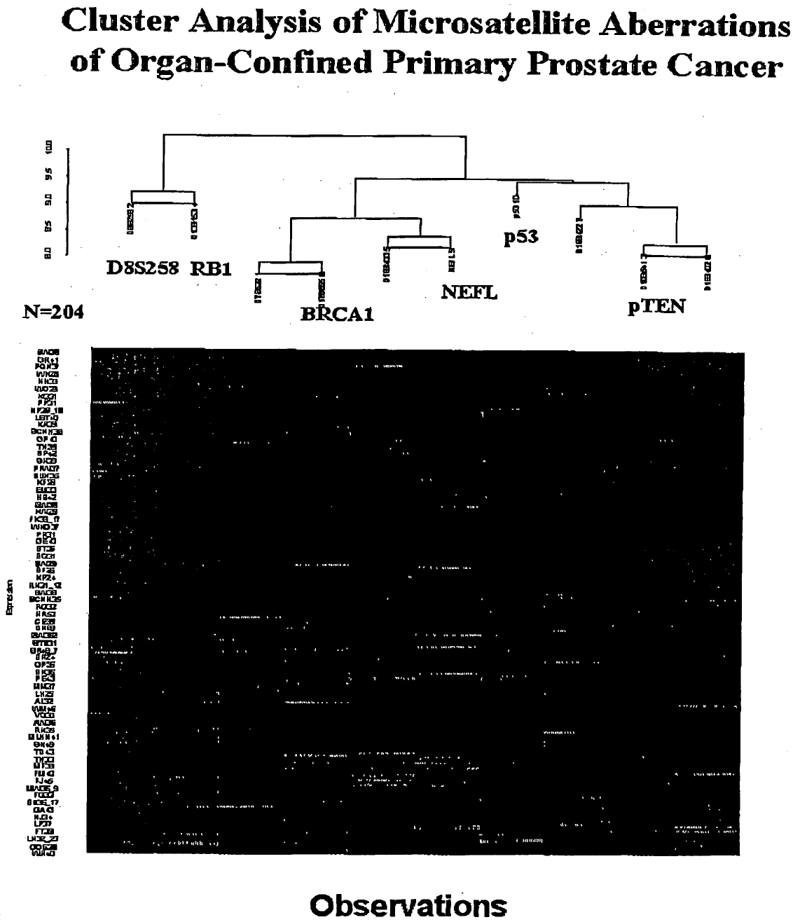

FIG. 1 shows in section 1 the cell diameter before the cells were suspended for density gradient centrifugation using a hyper-osmolarity buffer. The cell diameter is on average 8.02 μm. Compared with this, FIG. 2b shows the cell diameter after Immersion in a hyper-osmolarity buffer such as Nyoprep or Polymorphprep- The average diameter was reduced to 4.97 μm.

Below follows an exemplary description of the cell isolation process for different types of cells and tumours.

I. Isolation of Cells Extracted from a Breast and Ovary Carcinoma.

A. Preparation of Samples
1. Cell suspensions are prepared according to the method described In V. This suspension Is Incubated for 10 minutes at room temperature (RT) in a PABB buffer (saturates binding sites).
2. Centrifugation (10 minutes, 1500 rpm).
3. Re-suspend in 1000 μl PABB buffer, incubate for at least 30 minutes at RT on a horizontal shaker.
4. Add 20 μl ErbB-2 antibodies (Ab-2, anti-mouse for human ErbB2), incubate for at least 30 min. at RT
5. Add 2 ml 1% PBS/BSA, mix and centrifuge. Re-suspend In 70 μl PABB buffer and Incubate for about 15 min. at RT.
6. Add 10 μl antibodies linked to magnetic beads (IgG1 rabbit/mouse antibodies, 1:10 dilution), incubate for at least 30 min. at RT (20 μl for 10⁷ cells, incubation volume 80 μl).
7. Wash with 1 ml PABB buffer, re-centrifuge (10 min. 1500 rpm).
8. Re-suspend in 500 μl 1% PBS/BSA

B: Column

Use MS-Columns (Miltenyi Biotech GmbH), capacity 10⁷ cells
1. Wash column with 500 μl 1% PBS/BSA
2. The cell suspension (diluted with 1% PBS/BSA is added to the column and the negative fraction Is collected, rinse the column with 1.5 ml 1% PBSIBSA afterwards and collect as well.
3. Using a 10 ml one-way syringe and a three-way tap installed on top rinse about 3 ml PBS/BSA bubble free from below through the column so that cells pass the column again. Again, collect for a negative-control. Should the column clog, i.e. the flowing speed is reduced, rinse the column immediately with PBS/BSA from below and use a new column for the remaining sample material.
4. In order to collect the positive fraction, remove the column from the magnet and put it on to a further 10 ml tube.
5. Rinse the column with 1 ml 1% PBS/BSA and collect the fraction, fill the column again with 1 ml 1% PBS/BSA and press the positive fraction through the column.

C: Cytospins
1. The fractions are centrifuged and suspended in 1% PBS/BSA.
2. Prepare cytospins (8 min., 400 rpm) of positive and negative fractions.
3. Mark cell point on cytospin with a wax crayon, fix for 20 min. with 4% PFA, wash afterwards for 2×5 min. with 1% PBSIBSA, store at 4° C. In a moisture chamber.

II. Isolation of Tumour Cells from Blood Samples for the Characterisation of Mamma or Ovary Carcinoma.
1. Gradient separation as described under III.1
2. Prepare sample and saturated tumour cells as described under I.A, pre-supposing production of cell pellets as described under II 1

III. Isolation of Tumour Cells from Blood Samples for the Detection and Cell Characterisation of Prostate, Bladder, Colon and Gastric Carcinomas.

This is a detailed description of the procedure:
1. Gradient separation with Polymorphprep™ and Nycoprep™
2. Magnetic cell separation
   a) preparation
   b) columns
3. Cytospins for staining
4. Staining
   1: Gradient separation (here as example centrifugation with blood samples of a prostate carcinoma patient)
   a) Start with 3 ml Polymorphprep™ (gradient) (density 1.113, Nycomed)
   b) Carefully overlay with 3 ml Nycoprep™ (gradient) (density 1.068)
   c) Overlay without mixing with 4 ml EDTA patient blood
   d) Centrifuge for 30 min. at 1500 rpm
   e) Pipete serum (spread evenly)
   f) Transfer monocyte fraction including tumour cells (M) as well as leucocyte fraction (L) in PP tubes, divide into four PP tubes, mix (wash) with the double amount of PBS pH 7.4, centrifuge (20 min. 1500 rpm), discard supernatant.
   g) Discard erythrocyte fraction
   h) Fix monocyte pellet with a total of 2-5 ml PFA 4% (para-formaldehyde in PBS)
   i) Collect leucocyte pellet in a total of 1 ml PBS and transfer to two Eppendorf cups (collect)
   j) Centrifuge (Epifuge, 10 min. 1500 rpm), discard supernatant, freeze (−20° C.)
2. Magnetic cell separation using micro beads to increase concentration of tumour cells

For the following process only one pellet is used, the supernatant is discarded after each washing process.

Preparation:
a) centrifuge the pellet diluted with PFA, discard supernatant
b) prepare dilution buffer 1× (wash buffer PBS/BSA (35 ml H₂O_{deat}+4 ml dilution buffer 10×=dilution buffer 1×)
c) Add pellet to 5 ml PBS/BSA, then add to 35 ml dilution buffer/ concentration: 1 g/100 ml, add 5 ml permeability solvent for cells (P-Lsg), leave for 5 min. after shaking 1-2× otherwise the cells may possibly be destroyed). Divide on to three 15 ml PP tubes (Grelner Cell Star), centrifuge (10 min., 1200 rpm) without applying brake, discard supernatant
d) 5 ml fixation solvent for cells (F-Lsg) +45 ml PBS (marking solvent), remove 30 ml, use 1-5 ml of this to re-suspend the pellet, re-suspend the remaining 25 ml, transfer to two 15 ml PP tubes, centrifuge (10 min. 1200 rpm) discard supernatant
e) Re-suspend pellet in 10 ml fixing solution, centrifuge (10 min. 1200 rpm), discard supernatant
f) Re-suspend pellet in 600 μl fixing solution
g) Add 200 μl blocking reagent (for PABB) and mix
h) Add 200 μl immuno magnetic pellets (e.g. MACS bead anti-mouse AK or for example anti-rabbit, -goat, -sheep, -pig and mix (cytokeratine making for staining)
i) Incubate for 45 min. at RT
j) Add 4 ml fixing solution, mix, centrifuge (10 min. 1200 rpm), discard supernatant
k) Add pellet to 1 ml PBS/BSA, fix for storing In about 1 ml PFA 4%

Magnet Separation
l) Centrifuge the monocyte fraction fixed in 4% PFA (Including tumour cells), 10 min., 1200 rpm, discard supernatant
m) Saturate column with 3 ml PABB (rinse from top with pipette)
n) Add pellet to initially at least 12 ml PBS/BSA (adjust Individually depending on pellet size), dilute gradually starting with 3 ml in PP tube, then mix and gradually transfer to column (add further dilutions directly to column), separate negative fractions as negative controls. The flowing speed must be constant.
o) Using a one-way syringe with a three-way tap fixed to the top rinse about 3 ml PBS/BSA bubble free from bottom to top through the column, so that cells can pass the column again. Again, save negative controls. Should one column clog, i.e. the flowing speed is reduced, rinse the column immediately with PBS/BSA from bottom to top and use a new column for the remaining sample material.
p) Rinse the column gradually with a further 5-10 ml PBS/BSA and save as negative controls (all negative controls should by now have been rinsed off). The positive cells should have remained In the column. They have become magnetized because of the anti-cytokeratine antibody and a magnetizeable pellet.
q) Rinse the column again from bottom to top at a slow flowing speed with PBS/BSA
r) Remove the column slowly from the magnet and rinse the positive fraction (+) first of all without plunger with 5-6 ml PBS/BSA.
s) Fill the column again with 3-4 ml PBS/BSA and press the positive fraction (++) through the column using the plunger.
PABB: 5 ml AB serum 10% (v/v)+50 μl BSA-C 0.1% (v/v)+45 ml PBS
PBS/BSA: 1 g BSA In 100 ml PBS, pH 5.4=->1% (v/v) 0.1% triton: dilute triton ×100 with PBS pH 7.4 1:1000

Usually, In the first positive fraction (+) the ratio tumour cells/monocytes is clearly lop-sided, frequently there are only a few monocytes and therefore a high degree of purity of tumour cells.
t) Centrifuge and add cell pellet to 1-2 ml PBS

3. Cytospins for Staining
a) Transfer 200 μl diluted sample/spin to microscopic slide (pipette, filter, funnel, empty reference): also +- and ++-controls and negative (−) controls to another slide, two of each for controls in case the staining does not work. Label slides (+, ++, −) date, name of patient, comment, “prior to magnet separation”)
b) Centrifuge for 8 min. (400 rpm)
c) Mark spot with a wax crayon, cells on slides are fixed with 30 μl PFA/spot for about 20 min.
d) Wash for 10 min. with 30 μl PBS/BSA per spin, shake off surplus fluid, store at 4° C.
e) Centrifuge the remaining diluted samples (10 min. 1000 rpm), discard supernatant, add pellet to about 2 ml PFA for fixation and store at 4° C.
4. Staining with streptavidin, a with antibodies which are conjugated with fluorescent pigments (Alexa-488/584, FITC, RPE, etc.)
a) the pellets mounted on slides (see last point in 3. Cytospin) are made permeable with 0.1% triton per spin for 10 min. In a (Fauchtekammer)
b) saturate for 20 min. with PABB
c) 15 ul secondary antibody (Alexa 594 streptavidin) per spin for 30 min. (dilute 1:100 with PABB in Eppendorf cups, e.g. 396+4/99+1 etc.), shake off surplus
d) wash for 2×5 min each (or 1×10 min) with 30 μl PBS/BSA per spin, shake off surplus
e) saturate for 20 min. with 20 μl PABB, shake off surplus
f) add 15 μl PSA (antibody DP033, dilute 1:100) for 45 min., shake off surplus
g) wash for 10 min. with PBS
h) 15 μl secondary antibody (Alexa 488 goat anti rabbit 11088, dilute 1:100) for 30 min. incubate, shake off surplus
i) wash with PES for 10 min.
j) wash with 30 μl H₂O_{bidest} for 30 min., shake off surplus

IV. Isolation of Tumour Cells from Urine Samples

The following steps were taken to achieve this:
1. Collect urine sample
2. Determine density and quantity (ml), centrifuge and determine pellet size
3. Cytospins for PAP staining
4. PAP staining
5. If required: magnetic separation
   a) preparation
   b) columns
6. Cytospins for antibody staining
7. Cytokeratine staining
   1. collection of sample
   2. determine density and quantity
      a) transfer urine to PP tubes
      b) transfer urine to graduated cylinder, determine density, write extra protocol
      c) centrifuge In PP tubes (10 min. at 1000 rpm), discard supernatant
      d) estimate pellet size
      e) add pellet to about 2 ml PFA (fixation) for storage, use more PFA depending on pellet size
   3. Cytospins for PAP staining
      a) dilute pellet with PBS/BSA (no clouding), start with about 3 ml, depending on size of pellet
      b) transfer 200 μl diluted sample/spin to slide (pipette, filter, funnel)
      c) centrifuge for 8 min. (400 rpm) 2×g
      d) examine whether cells lie separately on slide, otherwise dilute further and repeat step c)
      e) fix cells to slide (mark generously with wax crayon), 30 μl PFA/spin (pipette) for 20 min. (close lid), remove surplus (abklopfen), cool storage or proceed (point 4)
      f) centrifuge the remaining samples (10 min. 1000 rpm), discard supernatant, fix pellet in about 2 ml PFA (para-formaldehyde+formalin) and store at 4° C. PFA: paraformaldehyde +37% formalin 1:10 or dilute with PBS PBS/BSA: 1 g BSA (bovine albumine)/100 ml PBS
   4. PAP staining and first evaluation (control stain)
      a) stain cells on slide according to the method of Papanicolaou or use other control stains
   5. Magnet-associated cell sorting, corrected with magnetic micro beads on anti-mouse antibodies

Always work with the pellet only and discard the supernatant after each washing process.

Preparation
a) centrifuge the pellet which had been diluted with PFA, discard supernatant, add 2 ml/PBS/BSA to the pellet, centrifuge (7×g 10 min. about 900 rpm), remove supernatant
b) prepare dilution buffer (washing buffer) (36 ml H₂O_{dest}+4 ml dilution buffer 10× dilution buffer 1×)
c) Add first 5 ml PBS/BSA to pellet and then 35 ml dilution buffer, followed by 5 ml P-solution, leave for 5 min. shake 1-2× beforehand, divide on to 3 15 ml PP tubes (e.g. Grelner Cell Star), centrifuge (10 min. 900 rpm), discard supernatant
d) 5 ml F solution (corresponds to PABB, +45 ml PBS (=marking solution), remove 30 ml, use 1-5 ml of this to liquefy the pellet, put into a tube, suspend with the remaining 25 ml, transfer to 2 15 ml PP tubes, centrifuge for 10 min. at 900 rpm, discard supernatant
e) Re-suspend pellet in a total of 10 ml F-solution, centrifuge again (10 min., 900 rpm) discard supernatant
f) Add pellet to 500-1000 μl (minimum 200 μl) diluted cytokeratine 7, dilute 1:50 with PABB (e.g. 490 μl PABB +10 ul cytokeratine 7), incubate for 30 min., centrifuge (10 min., 900 rpm), remove supernatant
g) Wash with 5-10 ml PABB, centrifuge (10 min. 900 rpm), discard supernatant
h) Re-suspend pellet in 600 μl F-solution
i) Add 200 μl undiluted FCR blocking reagent and mix
j) Add 200 μl undiluted magnetic micro beads conjugated with anti-mouse-antibody and mix
k) Incubate for 45 min. at RT
l) Add 4 ml F-solution, mix, centrifuge (10 min, 900 rpm) discard supernatant
m) Add pellet to about 1 ml PBS/BSA, store cells in about 1 ml PBS 4% at 4° C.

Modified cell separation using separation columns
a) centrifuge the monocyte fraction (including tumour cells) which was first fixed in 4% PFA or dissolved in PBS/BSA (10 min. 900 rpm), discard supernatant
b) saturate the column with 3 ml PABB (add PABB with a pipette from the top of the column)
c) Dilute the pellet In PBS/BSA gradually, starting with 10 ml in PP tubes, mix, gradually add to the column, collect suspension as negative control
d) Use a 10 ml one-way syringe with a three-way tap fixed to it to rinse about 3 ml PBS/BSA bubble free from below through the column so that the cells pass the column again.
e) Rinse the column gradually with a further 5-10 ml PBS/BSA and collect the negative controls (all negative cells should now have been rinsed off and the positive cells caught in the column. Cells that are magnetisable by anti-cytokeratin antibodies and magnetic micro beads are caught in the column.
f) Rinse the column again at slow speed from bottom to top with PBS/BSA
g) Remove the column carefully from the magnet, rinse the first positive fraction (+) using 5-6 ml PBS/BSA from the column in to a PP tube.
h) Fill the column again with 34 ml PBS/BSA and push this through the column with a plunger. This is the second positive fraction (++) which is collected in a second PP tube.
   As a rule, and in comparison to the first positive fraction (+), the second fraction (++) contains only tumour cell and very few separate leucocytes, erythrocytes and urothelial cells
i) centrifuge (10 min. at 900 rpm), discard supernatant
6. Cytospins for antibody staining
a) dilute pellets (see 5) PBS/BSA with 400 μl to 2000 μl, depending on the size of the pellet
b) transfer 200 μl diluted positive fraction to slide by means of cytocentrifuge. Centrifuge negative controls (−) to a microscopic slide
c) conditions for centrifugation: 8 min (set time 8/enter ), 400 rpm=2×g (set speed 40/enter, start)
d) Fix cells to slide using 30 μl PFA per spot for20 min in a moisture chamber, remove surplus afterwards and if applicable store at cool temperatures
7. Staining of cytokeratine

Reagents
4% PFA (dilution: para formaldehyde=37% formalin+PBS 1:10)
0.1% triton×100 (in PBS)
10% AB serum (5 ml AB serum Biotest AG+50 μl BSA-c+45 ml PBS/BSA)
1% PBS/BSA
first antibody: mouse anti cytokeratin antibody. Working concentration 1:50 (in PABB) (C7, C20 or PAN)
secondary antibody: anti mouse Alexa 594 antibody. Working concentration 1:100 (in PABB). Isotype control in 10% AB serum

If applicable In prostate carcinoma patients:
first antibody: anti PSA antibody working concentration 1:40 (in PABB)
second antibody: anti rabbit Alexa 488 antibody working concentration 1:100 (in PABB)
a) the pellets fixed to the slide (see last point of Cytospin) are made permeable using 30 μl 0.1% Triton ×100 per spin for 10 min., remove water solution afterwards,
b) block with 30 μl 10% PABB serum for 20 min., remove water solution afterwards,
c) 15 μl diluted first antibody (mouse anti cytokeratin antibody suitable for specificity C7, C20, PAN) per spin for 30 min., remove water solution afterwards
d) wash with 30 μl PBS/BSA per spin for 10 min., remove, repeat twice
e) 15 μl diluted secondary antibody (anti mouse Alexa 594, antibody) per spin for 30 min. remove water solution afterwards,
f) wash with 30 μl PBS/BSA per spin for 10 min., remove water solution afterwards, repeat twice

V. Preparation of Cell Suspensions from Solid Human Tumour Tissue (e.g. Mamma or Prostate Carcinoma Tissue)

Reagents:

Invasive medium (Dulbecco's modified eagle medium)

1% (v/v) 2 mM Lglutamine

1% (vlv) antibiotic/antimycotic solution

0.1% (w/v) bovine serum albumine (BSA) in invasive medium Trypan blue solution (0.4%

(Gew/Vol) Sigma, Deisenhofen

Procedure:

Tissue samples from mamma carcinoma, benign breast tumours or prostate carcinoma are collected during the operation and put Into sterile tubes with a standard medium and put on ice until disaggregation (4 hours after sample collection at the latest). About half of each tissue sample is saved for later expression analysis and conserved in fluid nitrogen. The other half is disaggregated mechanically using a Medimachine (Dako, Hamburg). For the disaggregation the mamma tissue is cut with a scalpel into 3-10 mm² pieces and put into a Medicon together with 1.5 ml invasive medium. The tissue is disaggregated In the Medimachine within 2-3 min. to a cell suspension which contains separate cells and cell aggregates (cell clusters) of up to about 30 cells. The cells are counted microscopically using a Neubauer cell chamber. The number of live-cells Is determined with a Trypane blue exclusion test which works on the basis that certain pigments cannot reach the cell nucleus, whereas dead cells will absorb this pigment (Kaltenbach et al. 1958: Lindl und Bauer, 1994).

The process of cell isolation described above is followed by the genotyping of isolated cell clusters in order to allocate them to areas within the primary tumour by means of PCR.

The following is an exemplary description of nucleinic acid isolation in different cell materials

1. DNA of Cell Clusters Obtained from Peripheral Blood, and of Tissue Samples which were Micro-Dissected, Rendered Free of Paraffin, Fixed and Dyed.

PSA- and cytokeratine-positive tumour cells and tumour cell dusters, as well as normal monocytes as negative controls or as reference for LOH calculation, are micro-dissected with a fine sterile needle, using an inverse light microscope (Leitz Diavert). They are then each transferred to 1.5 ml sterile reaction vessels (Eppendorf Biopure). This method can also be used to micro-dissect foci of small multi-focal tumour areas from already stained and pathologically examined sections. Depending on the number of cells (about 50-1000 cells) the cells are added to 10-200 μl LTE buffer (10 mM Tris/HCl, 1 mM EDTA. pH 7.5) and Incubated with 1-20 μl proteinase K (>600 mAU/mL) in a thermo pack or water bath at 50-60° C. for 1-10 h and put on ice afterwards for 5 min. Following this the samples are centrifuged for 1 min. at 10.000 rpm. The samples are then diluted to a 70% solution using 99.8% ethanol p.a. (Roth). Following a short vortex interval the samples are centrifuged at 15.000 rpm for 20 min., the supernatant discarded and the DNA-pellet dried at room temperature. The DNA is re-suspended in 10-200 μl Al LTE buffer or twice distilled water, incubated at RT for 1 hour (re-hydration) and stored at −20° C. until used for PCR.

2. DNA Obtained from Preserved Tumour Tissue of Tumours with One or Several Foci

The DNA isolation from fresh or formaline-preserved tissue or in paraffin immersed tissue of primary tumours with one or several foci Is performed according to the protocol of the commercially available QIAmp DNA Mini kit (Qiagen, Hilden) or any comparable system made by another company. This kit contains QIAmp DNA mini spin columns, proteinase K for the proteolytc digestion of tissue, lysis buffer AL and ATL, ethanol containing wash buffer AW1 and AW2 and the elution buffer AE. Fresh primary tumour tissue is processed either mechanically with a scalpel or a tissue shredder (e.g. Medimachine, DAKO). Tissue sections immersed in paraffin are put into 100% xylol for removal of the paraffin before the DNA Isolation. To do this the samples are Incubated for 1 h at 70° C. In 1 ml xylol in 1.5 ml Eppendorf reagent vessels in a commercially available thermal block. Centrifuge for 3 min., discard supernatant, repeat this procedure twice. Wash the tissue three times with 99.8% ethanol (Roth), dry and put into a lysis buffer. Then follows the isolation according to the manufacturer's protocols.

The DNA isolation from EDTA ant coagulated full blood is performed with the QIAmp DNA blood mini kit (Qiagen, Hilden) following the known protocols or comparable procedures of other manufacturers.

If required for cell lyses, the full blood is incubated in a thermal block for 10 min. at 54° C. with buffer AL and proteinase K. It is then mixed with ethanol and applied to a column (QIAmp DNA Mini Spin Column). The samples are washed with AW1 and then AW2 and eluted with an elution buffer. For concentration measurement the DNA solution is measured on a photometer at 260, 280 and 320 nm, adjusted to 10 ng/μl and frozen at −20° C.

3. Isolation of RNA from Cells Isolated from Peripheral Blood

PSA- and cytokeratin-positive tumour cells and tumour cell clusters are micro-dissected with a fine sterile needle, using an inverse light microscope (Leltz Diavert). They are then put Into a 1.5 ml sterile reaction vessel (Eppendorf Biopure). This RNA isolation procedure strictly follows the protocols of the RNeasy Purification Kit for total RNA mini preparation (Qiagen, Hilden). This consists of; RNeasy Mini Spin Columns, collection tubes, 1.5 and 2 ml, buffer RTL, buffer RW1, buffer RPE and RNase free water.

The following is an exemplary description of the detection of carcinoma-specific, genetic changes and mRNA expressions by means of microsatellite PCR, multiple microsatellite PCR and TaqMan™ RT-PCR, in accordiance with the innovation presented here.

4. Microsatellite and Multiplex Microsatellite PCR

Three multiplex PCRs are used in total, consisting of microsatellite combination no. 1: D7S522, D8S258, D16S400; no. 2: NEFL, D138153, D178855 and no. 3: D108541, D16S402, D16S422. The principle of these PCR lies in the co-amplification of different DNA sections in one reaction vessel. The primer sequences are set in such a way that in the capillary electrophoretic separation no overlapping of length occurs in the amplification products. All primers are marked with fluorescent pigments which are activated at 488 nm (see table 3). All other commercially available fluorescent markers may also be used. Further, the PCR reaction conditions for 10 CA-repeats with the EGFR gene and a CA-repeat within the p53 gene were newly developed and optimised.

All PCRs can be performed on commercially available 0.2 ml or 0.5 ml reaction vessels or in 96 well trays of different manufacturers (e.g. Eppendorf, Hamburg) on an Eppendorf Mastercycler, Eppendorf Mastercycler Gradient (Eppendorf, Hamburg), a Gene AmpR PCR System 9700 (PE Applied Biosystems, Weiterstadt), or a commercially available, comparable thermocycler of other manufacturers.

The reaction volume can vary from 12 μl to 100 μl. The PCR reaction mixture consists of 5U/100 μl Ampliaq Gold™ or a polymerase of comparable quality (well proven are hot start polymerases), 1× GeneAmp^{R} dNTP, 2 mM MgCl2, 30 pM of each primer, 200 μM GeneAmp^{R} buffer (all reagents by PE Applied Biosystems, Weiterstadt) and 500 pg to 200 ng genomic DNA. The following temperatures are run for the PCR: Starting temperature for denaturation, 95° C., followed by 30-45 cycles consisting of a 95° C. denaturation phase for 30s, a 56° C.-62° C. annealing step (depending on the primer, all multiplex PCRs are uniformly performed at 56° C.) and an elongation step at 72° C. Following these cycles there is a 7 min. extension step at 72° C. The samples are then cooled down to and stored at 4° C.

The microsatellite p53 is processed in the same way as the multiplex PCRs (see table 2).

The microsatellite analysis is performed on a four colour laser-induced fluorescence capillary electrophoresis system, ABI Prism 310 Genetic Analyser or ABI 3700 DNA analyser (PE Applied Biosystems, Weiterstadt) or another comparable genetic analyser of another manufacturer. As separating medium the polymeres POP4, POP5, POP6 are used which are appropriate for the systems used. As standard for length the Genescan 500 TM TAMRA 500 can be used, or a comparable standard for length which is suitable for the capillary electrophoresis systems mentioned above. Analysis and evaluation was performed with the Genescan software.

| Reagents: | volumes: | |
|---|---|---|
| Water | bis zu 25 | μl |
| 10 * PCR buffer II (PE) | 2.5 | μl |
| 25 mM MgCl₂ solution (PE) | 2 | μl |
| dATP, 10 mM (PE) | 0.25 | μl |
| dCTP, 10 mM (PE) | 0.25 | μl |
| dGTP, 10 mM (PE) | 0.25 | μl |
| dTTP, 10 mM (PE) | 0.25 | μl |
| 5′-3′ Primer 10 μM | 0.5 | μl |
| 3′-5′ Primer 10 μM | 0.5 | μl |
| AmpliTaq Gold (PE) or | 0.25 | μl |
| AmpliTaq DNA Polymerase | 0.25 | μl |
| Total: | 25 | μl |

TABLE 2
Temperature cylces for microsatellite PCR: for D7S2429, BB1/2,
CAII, D7S2550, CAIII, CAIV, CAVI, D7S2467, D7 D7S2552:

| | |
|---|---|
| 95° C. - 4 Min. | |
| 62° C. - 1 Min. | |
| 72° C. - 1 Min. | |
| | 95° C. - 1 Min. |
| 31 cycles | 62° C. - 1 Min. |
| | 72° C. - 1 Min. |
| 72° C. - 8 Min. | |
| 4° C. - Indefinitely | |
| for D7S494: | |
| 95° C. - 4 Min. | |
| 58° C. - 1 Min. | |
| 68° C. - 1 Min. | |
| | 95° C. - 1 Min. |
| 31 cycles | 58° C. - 1 Min. |
| | 68° C. - 1 Min. |
| 68° C. - 8 Min. | |
| 4° C. - indefinitely | |
| for D7S499: | |
| 95° C. - 4 Min. | |
| 56° C. - 1 Min. | |
| 68° C. - 1 Min. | |
| | 95° C. - 1 Min. |
| 31 cycles | 56° C. - 1 Min. |
| | 68° C. - Min. |
| 68° C. - 8 Min. | |
| 4° C. - indefinitely | |

TABLE 3
Sample preparation ABI Prism 3700: Fragment analysis (GeneScan)
Pipetting scheme:

| Reagents: | Volume: | |
|---|---|---|
| Length standard for capillary electrophoresis | 0.5 | μl |
| e.g. GeneScan Tamra 500 | | |
| HPLC-water oder formamide | 18.5 | μl |
| PCR-product | 1 | μl |

Standard: GeneScan-500 TAMRA Size Standard PE Biosystems
Denaturing of samples: thermo block for 2 min. at 80-90° C.

TABLE 4

Sequence of primers, gene loci and fragment
length of PCR products

| | | Cytoge- | Frag- |
|---|---|---|---|
| | | netische | ment- |
| Primer- | | Lokall- | range |
| locus | Primersequenzan | sation | [bp] |
| D7S522 | 5′Fam-GCA GGA CAT GAG ATG | 7q31.1 | 116- |
| | ACT GA-3′ | | 126 |
| | 5′-GTT ATG CCA CTC CCT CAC | | |
| | AC-3′ | | |
| BB1 + 2 | 5′-Fam-GTT TGA AGA ATT TGA | 7p12 | 114- |
| (EGFR) | GCC AAC C-3′ | | 128 |
| | 5′-TTC TTC TGC ACA CTT GGC | | |
| | AC-3′ | | |
| CAII | 5′Fam-CT CGA GGT CTC ATC | 7p12 | 164- |
| (EGFR) | CTC TTT CC-3′ | | 168 |
| | GCA GAG GTG CAC AAA GGA | | |
| | GTAA-3′ | | |
| CAIII | 5′-Fam-AG GCC CAC AGA GGA | 7p12 | 117- |
| (EGFR) | GAT AAC AG-3′ | | 121 |
| | 5′-CAG GTG TGG TAG ATG CCA | | |
| | AAG A-3′ | | |
| CAIV | 5′-Fam-GC AAC TTA TCC AAA | 7p12 | 184- |
| (EGFR) | CCC TGA CC-3′ | | 204 |
| | 5′-AGA GTG GAC TAG GAA ATG | | |
| | CTA GGA G-3 | | |
| CAIV | 5′-Fam-AG TTC CTG ACT GGG | 7p12 | 151- |
| (EGFR) | AAT TCG AT-3′ | | 155 |
| | 5′-TTG GCC AAA TTA CAC ACC | | |
| | TTT G-3′ | | |
| D7S2550 | 5′-Fam-TTC CAT TTG TCT CGG | 7p12 | 266- |
| (EGFR) | TT-3′ | | 278 |
| | 5′-AGT CTC CTC GTC TCA CAC | | |
| | CT-3′ | | |
| D7S2429 | 5′-Fam-CAG TGC TGG AGT TGT | 7p12 | 170- |
| (EGFR) | TCA AG-3′ | | 180 |
| | 5′-CTG GGA GTC AAG TGT TTT | | |
| | GG-3′ | | |
| D7S2467 | 5′-Fam-TGC TAA GTC TTG ATT | 7p12 | 238- |
| (EGFR) | TTG CC-3′ | | 244 |
| | 5′-AAC GGT CAT CTG TGT TCG- | | |
| | 3′ | | |
| D7S478 | 5′-Fam-GGT GTT TGT GTC ATT | 7p12 | 312- |
| (EGFR) | ACG CT-3′ | | 314 |
| | 5′-TTT GCT GTA GAG GAT GCA | | |
| | AT-3′ | | |
| D7S870 | 5′-Fam-TTC GGG CTC TCT GTT | 7p12 | 136- |
| (EGFR) | ATA AA-3′ | | 152 |
| | 5′-CCG AAG CAG GAT TTT ATT | | |
| | TC-3′ | | |
| D8S258 | 5′-Fam-AGC TGC CAG GAA TCA | 8p22 | 216- |
| | ACT GAG AG-3′ | | 230 |
| | 5′-GAT GCT CAC ATA AAG GAG | | |
| | GGA GG-3′ | | |
| NEFL | 5′-Fam-CC AAT ACC TGC AGT | 8p21 | 97- |
| | AGT GCC-3′ | | 105 |
| | 5′-GAG CTG CTT AAC ACA TAG | | |
| | AA-3′ | | |
| D10S541 | 5′-Fam-CAC CAC AGA CAT CTC | 10q14.2 | 153- |
| (PTEN) | ACA ACC-3′ | | 175 |
| | 5′-CCA GTG AAT AGT TCA GGG | | |
| | ATG G | | |
| D13S153 | 5′-Fam-AG GGT TAT GTA TAA | 13q14.2 | 170- |
| (Rb1) | CCG ACT CC-3′ | | 190 |
| | 5′-Fam-GTC TAA GCC CTC GAG | | |
| | TTG TGG-3′ | | |
| D16S400 | 5′-Fam-GGT TCA CAA TTG GAC | 16q22.2- | 165- |
| | AGT AT-3′ | 23.1 | 179 |
| | 5′-GAA CCC TCC ATG CTG ACA | | |
| | TT-3′ | | |
| D16S402 | 5′-Fam-GT ACC CAT GTA CCC | 16q24.2 | 110- |
| | CCA ATA-3′ | | 120 |
| | 5′-CAA AGC ACC ACA TAG ACT | | |
| | AA-3′ | | |
| D16S422 | 5′-Joe-GAG AGG AAG GTG GAA | 16q24.2 | 105- |
| | ATA CA-3′ | | 129 |
| | 5′-GTT TAG CAG AAT GAG AAT | | |
| | AT-3′ | | |
| P53 | 5′-Fam-AAG AAA TTC CCA CTG | 17p13.1 | 132- |
| | CCA CTC-3′ | | 148 |
| | 5′-ATC CCC TGA GGG ATA CTA | | |
| | TTC-3′ | | |
| D17S855 | 5′-Fam-GG ATG GCC TTT TAG | 17q21 | 139- |
| (BRCA1) | AAA GTG G3′ | | 153 |
| | 5′-ACA CAG ACT TGT CCT ACT | | |
| | GCC-3′ | | |

5. TaqMan RT-PCR

PSA- and cytokeratin-postive tumour cells and tumour cell clusters are micro-dissected on an Inverse light microscope (Leitz Diavert) and transferred to a 1.5 ml sterile reaction vessel (Eppendorf Biopure). The RNA isolation strictly follows the protocols of the RNeasy purification kit for total RNA mini preparation (Quiagen, Hilden). This consists of: RNeasy mini spin columns, collection tubes 1.5 and 2 ml, buffer RTL, buffer RW1, buffer RPE and RNase-freee water. The RNA is converted to cDNA In a two-tube-reaction. This procedure is in accordance to the protocols for the Omniscript Reverse Transcriptase Kit (Qiagen, Hilden). The reaction volume is 20 μl. The reaction mixture consists of RT 1× buffer, dNTPs (0.5 mM each), μM Oligo-dT primer. 10 units RNase inhibitor, 4 units omniscript reverse transcriptase and RNase-free water. For the RT-PCR, RNA samples are denatured at 65° C. for 5 min. and then put on ice.

RT-PCR can be performed on an Eppendorf Mastercycler, Eppendorf Mastercycler Gradient (Eppendorf, Hamburg), a Gene Amp R PCR System 9700 (PE Applied Biosystems) or another commercially available and comparable thermo cycler of other manufacturers.

The PCR process starts with a 37° C. incubation for 60 min., followed by a 93° C. denaturation. RNA isolation and RT-PCR can be performed with other commercially available methods which are suitable for small quantities of tissue. Suitable are for instance ExpressDirect™ kit for mRNA capture and RT system for RT-PCR (Pierce Rockford).

The real-time PCR can be measured on an ABI prism 9700 HAT sequence detection system (PE Applied Biosystems, Weilerstadt) on 96 or 384 well trays sealed with ABI PRISM™ optical adhesive covers (ABI, Foster City). The reactions are usually measured in double- or triplicate determinations following the TaqMan^{R}-PCR Instructions of PE Applied Biosystems (Weiterstadt). The reaction mixture consists of a TaqMan^{R} universal PCR master mix, plus 90 nM each of both PSA specific primer (forward 5′TTCACCCTCAGMGGTGACCA- TaqMan probe (5′-CCAGCGTCCAGCACACAGCATGA). The temperature gradient starts with 50° C. incubation for 2 min., followed by a 95° C. denaturation for 10 min. followed by 40-60 cycles consisting of a 95° C. denaturation for 15 sec. and a 60° C. amplification for 1 min. For sequence and evaluation the SDS software (PE Applied Biosystems, Weiterstadt) was used. Primer and TaqMan probe are available from various manufacturers.

In order to obtain a positive control and for establishing the TaqMan PCR, RNA was isolated from cells of an LNCaP cell culture. This is known to be expressible for PSA. Female lymphocytes were used as negative controls.

6. Examples for the association of circulating cells to areas of primary tumours

a) Examination of Individual Patients

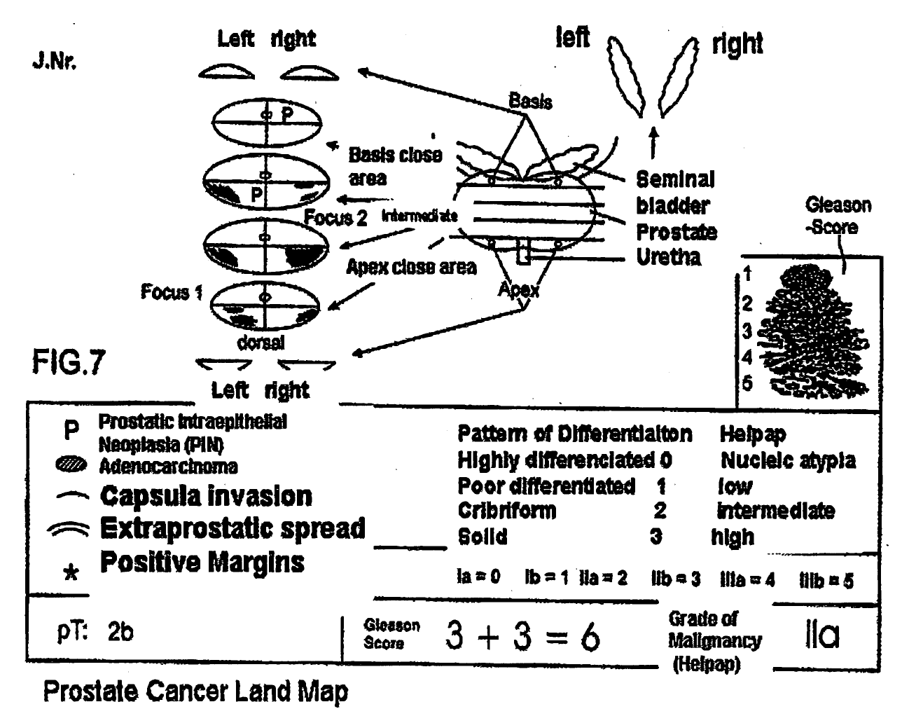

In individual patients the microsatellite markers in DNA were examined. The material used was PSA positive epithelial cells and separate foci of the primary tumour gained from the DNA of peripheral blood. The microsatellite marker were determined from DNA, following the protocol (described under point 4). DNA were obtaining according to protocols 1 and 2. Prostate tissue samples were systematically prepared according to the procedure described by Schmid et al. (Schmid H P et al., Akt Urologie 1993). A detailed mapping of the extent of the carcinoma was laid. Following colour marking of the edge of the operative cut, a documentation was produced of the tumour size, position of the tumour In relation to the pseudo capsule of the prostate (infiltration or penetration of capsule) and the closeness to or the crossing of the operative cutting margins. Histologically prepared tumour tissue was gained from the paraffin-immersed material of the primary tumour. FIG. 7 shows a so-called tumour map which shows where the samples were taken.

The age of the patient, the stage of the tumour and its histological parameters are summarised in the following table:

| | | | | | | PSA, präop |
|---|---|---|---|---|---|---|
| Nr. | Alter | PT (92′) | N | Grading | Gleason-Score | (ng/ml) |
| 1 | 67 | 2c | 0 | 2a | 7 | 9, 9 |
| 2 | 66 | 3c | 1 | 3b | 9 | 10, 6 |
| 3 | 69 | 2c | 0 | 2a | 6 | 6, 9 |
| 4 | 67 | 3b | 0 | 2b | 7 | 9, 5 |
| 5 | 63 | 3a | 0 | 3a | 8 | 7, 8 |
| 6 | 61 | 3a | 0 | 2b | 7 | 22, 6 |

TABLE 5
Comparison of genetic alterations between different foci of primary tumours and
circulating tumour cell clusters gained from blood

| | Patient Nr. 1 (39) | | | Patient Nr. 2 (85) | | | Patient Nr. 3 (50) | | |
|---|---|---|---|---|---|---|---|---|---|
| | Circulating | | | Circulating | | | Circulating | | |
| Loci | cells | Focus 1 | Focus 2 | cells | Focus 1 | Focus 2 | cells | Focus 1 | Focus 2 |
| D17S855 | hom. | hom. | hom. | No | No | no | LOH | No | LOH |
| | | | | LOH | LOH | LOH | | LOH | |
| NEFL | hom. | hom. | hom. | LOH | LOH | LOH | hom. | Hom. | hom. |
| D13S153 | hom. | hom. | Hom. | No | No | LOH | no | no | No |
| | | | | LOH | LOH | | LOH | LOH | LOH |
| D16S402 | LOH | LOH | LOH | No | No | LOH | Kein | no | no |
| | A2 | A2 | A1 | LOH | LOH | | LOH | LOH | LOH |
| D16S422 | no | no | no | No | No | no | no | no | No LOH |
| | LOH | LOH | LOH | LOH | LOH | LOH | LOH | LOH | |
| D10S541 | LOH | LOH | LOH | hom. | Hom. | Hom. | no | LOH | No |
| | | | | | | | LOH | | LOH |
| D7S522 | LOH | LOH | no | hom. | hom. | Hom. | no | No | no |
| | | | LOH | | | | LOH | LOH | LOH |
| D16S400 | hom. | Hom. | Hom. | LOH | LOH | Kein | hom. | Hom. | Hom. |
| | | | | | | LOH | | | |
| D8S258 | LOH | LOH | LOH | hom. | hom. | hom. | no | LOH | No LOH |
| | A1 | A1 | A2 | | | | LOH | | |
| | Patient Nr. 4 (54) | | | Patient Nr. 5 (117) | | | Patient Nr. 6 (97) | | |
| | Zirkulierende | | | Zirkulierende | | | Zirkulierende | | |
| Loci | Zellen | Focus 1 | Focus 2 | Zellen | Focus 1 | Focus 2 | Zellen | Focus 1 | Focus 2 |
| D17S855 | Kein | Kein | Kein | Kein | Kein | Kein | Kein | Kein | Kein |
| | LOH | LOH | LOH | LOH | LOH | LOH | LOH | LOH | LOH |
| NEFL | Kein | Kein | Kein | Hom. | Hom. | Hom. | hom. | hom. | Hom. |
| | LOH | LOH | LOH | | | | | | |
| D13S153 | LOH | LOH | Kein | LOH | LOH | LOH | LOH | LOH | LOH |
| | | | LOH | | | | | | |
| D16S402 | Kein | Kein | Kein | LOH | LOH | LOH | Kein | LOH | Kein |
| | LOH | LOH | LOH | | | | LOH | | LOH |
| D16S422 | hom. | hom. | Hom. | LOH | LOH | LOH | LOH | LOH | LOH |
| D10S541 | hom. | Hom. | Hom. | LOH | LOH | LOH | Kein | LOH | Kein |
| | | | | | | | LOH | | LOH |
| D7S522 | Hom. | Hom. | Hom. | Hom. | Hom. | Hom. | hom. | hom. | Hom. |
| D16S400 | Hom. | Hom. | Hom. | LOH | LOH | Kein | hom. | hom. | Hom. |
| | | | | | | LOH | | | |
| D8S258 | LOH | LOH | LOH | Hom. | hom. | hom. | Kein | LOH | Kein |
| | | | | | | | LOH | | LOH |

hom. = Homozygot
LOH = Loss of Heterozygosity
A1 = Allel 1
A2 = Allel 2

This proves that by means of analysing microsatellite DNA circulating cells can be directly assigned to certain foci of primary tumours.

It is therefore also possible to determine at which stage of development the primary tumour is by examining tumour cell clusters circulating in blood. Hence it can also be determined how the disease will further develop or how effective therapeutic measures are.

b) Examination of a Patient Pool

The DNA of organ-restricted prostate carcinomas was examined in 204 patients, using the described method for the determination of changes in polymorphic DNA sequences. A linkage could be shown between the changes In a polymorphic marker and a function gene. Therefore, in prostate carcinomas the marker D7S522, p53. D8S522, NEDL, D10S541, D13S153, D16S400, D16S402, D16S422, D17S855 from six chromomosomal locations were examined. With the aid of a hierarchic-agglomerative cluster analysis, tumour groups were defined with specific MS mutations (pattern recognition) (FIG. 2).

The mathematical cluster analysis led to the definition of three subgroups with up to four specific DNA changes:

1. p53, D16S402 or D16S400 (n=10);

2. D8S258 and / or NEFL, D13S153, D16S402 (n=9)

3. D10S541, D7S522, D13S153, D16S400 (N=11).

A rather rare combination of p53 and D13S153 (n=6) was found In tumour patients with a significantly low age at onset of illness (X) in comparison to all other patients (X=59 years; STD=4; X=64 years; STD=4; p0.02. Most recidivation occurred in subgroup 3 (4/9).

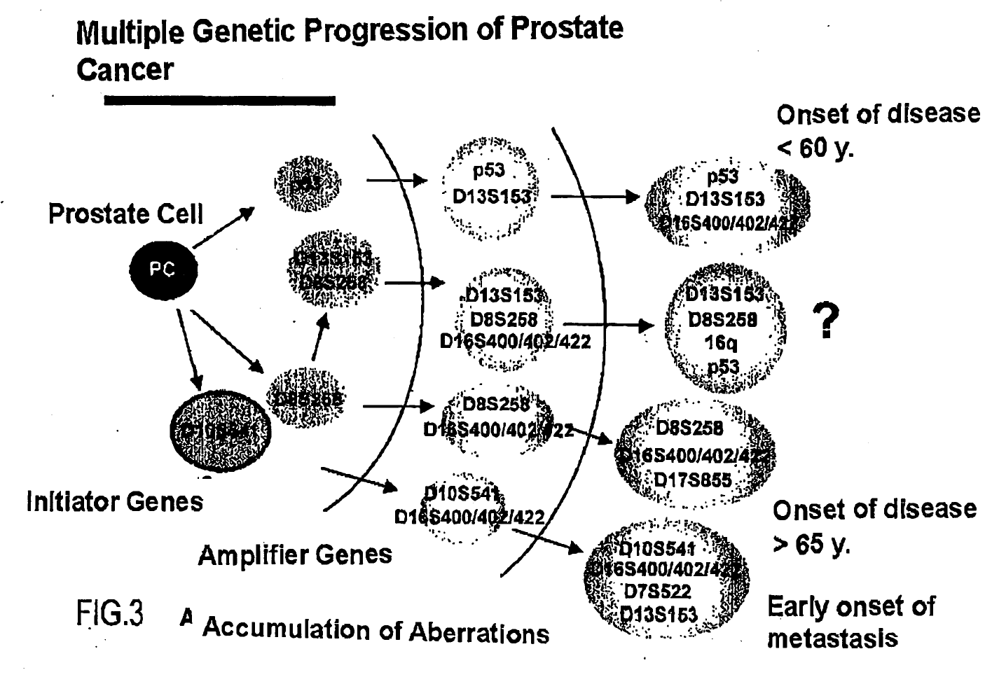

In summary, there are multiple paths of genetic development and progression of prostate carcinoma which can be Indicated using a combination of the examined markers (FIG. 3). Progression of a tumour is related to an absolute increase in DNA changes In polymorphic sequences. Even so, according to the study results presented here, there is a hierarchy of gene mutations which can be graded into clinically determinable subtypes of prostate carcinomas (FIG. 3). For example, p53, D10S541 and NEFL, D8S258 respectively, are primary mutations; mutations on chromosomes 16q and 13q however do not primarily initiate tumour growth.

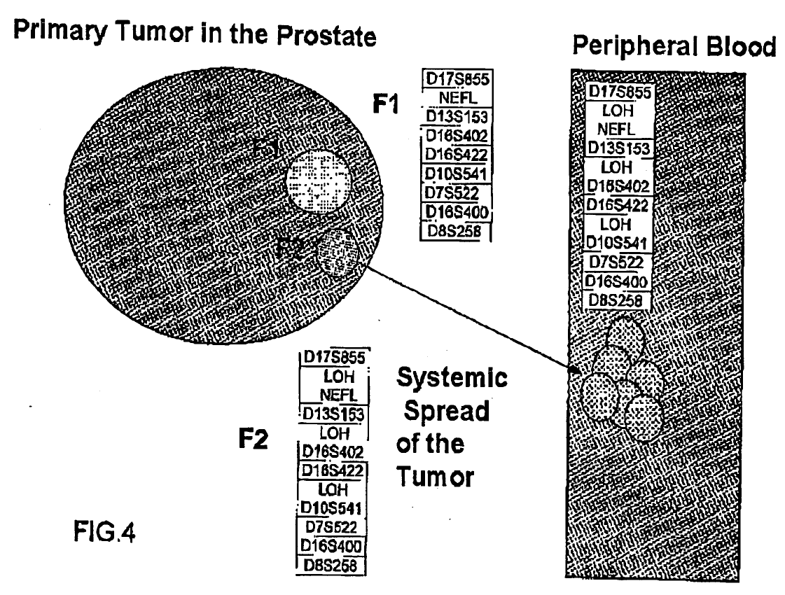

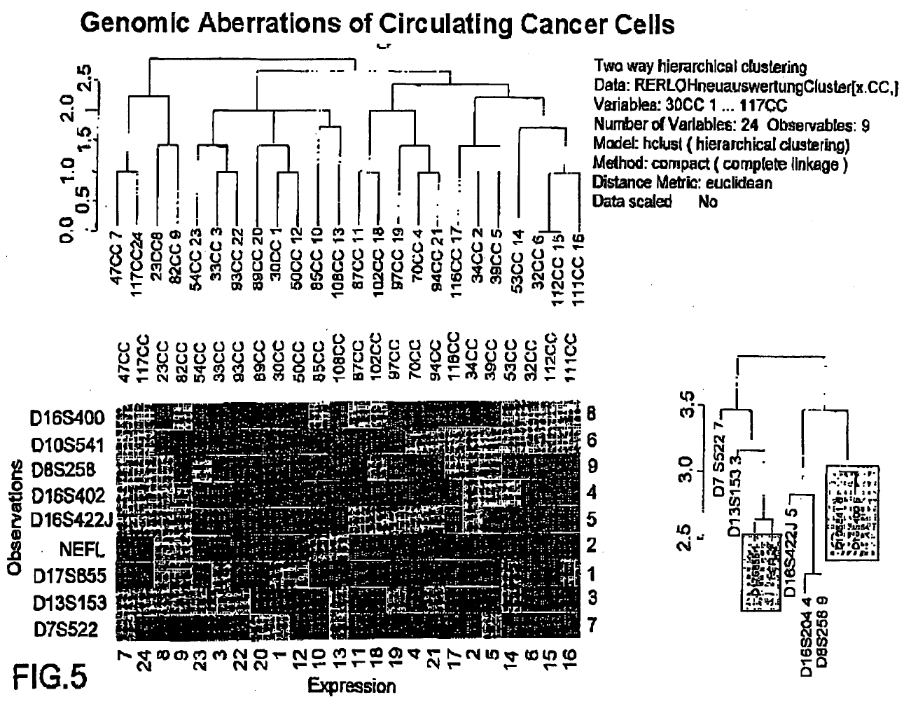

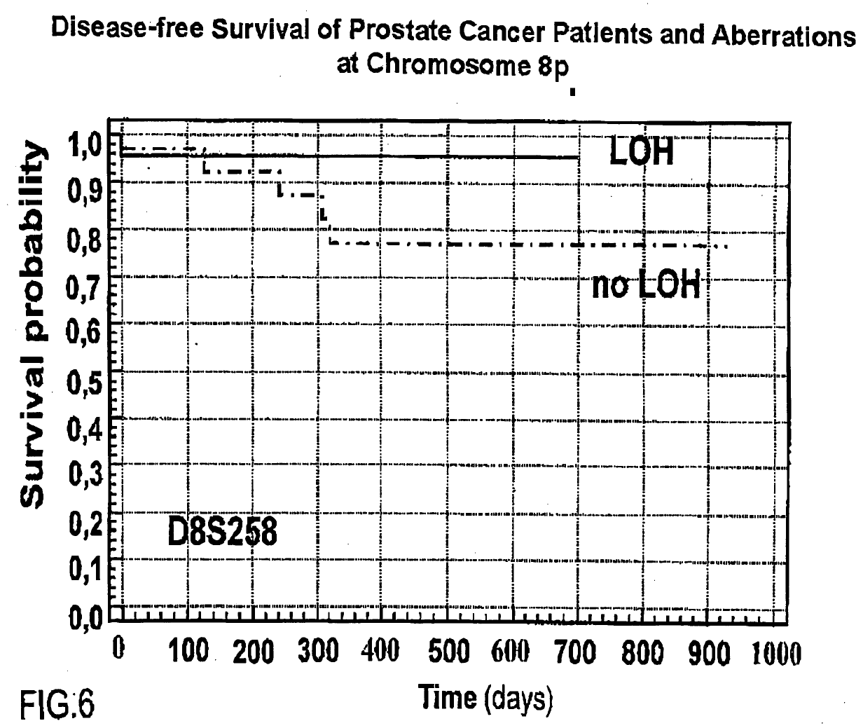

This hypothesis was applied to the comparison of changes in polymorphic DNA sequences between a primary tumour and circulating cells in 24 patients (FIG. 4). It was found that the release of tumour cells from the primary tumour is related to certain changes in the polymorphic DNA sequences (FIG. 5). The cluster with marker D10S541 which is related to an early formation of metastases Is also predominantly found In cell clusters of blood samples. On the other hand, alterations In marker D8S258 bear no relation to the spread of cells into peripheral blood. (FIG. 6). The evaluation of the intervals not affected by the disease shows that changes in this polymorphic marker are associated with a positive prognosis (FIG. 6).

c) Examples for the Detection of Prostate Carcinomas Through the Detection of Cell Clusters in Peripheral Blood of Patients and the Prognosis of the Result of a Prostate Biopsy

In 19 patents 50 ml blood was extracted before they underwent a transrectal-sonographic prostate biopsy. Cytokines and PSA-positive complete, small-cell cell clusters were isolated using the method described above.

In 8 patients the biopsy showed a prostate carcinoma (PCa), 11 patients had benign prostate tissue (BPH). The patients had the following serum PSA values and prostate volumes:

| | | PCa-Patients | BPH-Patients |
|---|---|---|---|
| Charakteristica | | (n = 8) | (n = 11) |
| t-PSA | Mittelwert | 9.0 (6.5) | 9.6 (4.3) |
| | (Stdabw.) Median | 6.9 | 8.6 |
| F/t-PSA | Mittelwert | 0.14 (0.07) | 0.15 (0.09) |
| | (Stdabw.) Median | 0.12 | 0.12 |
| Prostate vol. | Mittelwert(Stdabw.) | 45 ml (18) | 58 ml (15) |
| | Median | 46 ml | 60 ml |

| | | |
|---|---|---|
| CK+/PSA+-Zellhaufen | 6/8 | 3/11 |

t-PSA and f/t-PSA in serum do not allow a reliable prediction of the biopsy result. The examination of the cell dusters led to a correct prediction in 14 of 19 patients (efficiency of the test=74%).

1. Method for the detection and characterisation of primary tumours and separate areas of primary tumours, respectively, method comprising using sample material to isolate and concentrate cell clusters of tumour cells, followed by an analysis of the genetic changes in these isolated cell clusters.

2. Method according to the above claim 1, wherein the sample material consists of cell cultures, blood, urine, nipple aspiration fluid from the female breast or tissue from primary tumours.

3. Method according to claim 1, wherein polymorphic DNA of primary tumours or separate areas of primary tumours, and alterations therein, respectively, are recorded and compared with corresponding polymorphic DNA of cell clusters, and alterations therein, respectively.

4. Method according to claim 1, wherein DNA of the following polymorphic sequences are analysed: D7S522, D8S133, D8S258, D8S265, NEFL, D10S541, D10S1765, D10S579, D13S153, D16S400, D16S402, D16S413, D16S422, p53, BB1, BB2, CAII, CAIII, CAIV, CAV and/or D17S855.

5. Method according to claim 1, wherein the polymorphic DNA is reproduced before analysis.

6. Method according to claim 5, wherein the polymorphic DNA of three polymorphic sequences, D7S522, D8S256, D16S400 or NEFL, D13S153, D17S855 or D10S541, D16S402, D16S422 are analysed together and/or reproduced.

7. Method according to claim 6, wherein the polymorphic DNA is reproduced prior to analysis by polymerase chain reaction (PCR).

8. Method according to claim 7, wherein the polymorphic DNA is reproduced by using the following primer pairs:
GCAGGACATGAGATGACTGA
and
GTTATGCCACTCCCTCACAC
(for D7S522);
GTTTGAAGAATTTGAGCCAACC
and
TTCTTCTGCACACTTGGCAC
(for BB1 + 2);
CTCGAGGTCTCATCCTCTTTCC
and
GCAGAGGTGCACAAAGGAGTAA
(for CAII);
AGGCCCACAGAGGAGATAACAG
and
CAGGTGTGGTAGATGCCAAAGA
(for CAIII);
GCAACTTATCCAAACCCTGACC
and
AGAGTGGACTAGGAAATGCTAGGAG
(for CAIV);
AGTTCCTGACTGGGAATTCGAT
and
TTGGCCAAATTACACACCTTTG
(for CAV);
TTCCATTTGTCTCGGTT
and
AGTCTCCTCGTCTCACACCT
(for D7S2550);
CAGTGCTGGAGTTGTTCAAG
and
CTGGGAGTCAAGTGTTTTGG
(for D7S2429);
TGCTAAGTCTTGATTTTGCC
and
AACGGTCATCTGTGTTCG
(for D7S2467);
GGTGTTTGTGTCATTACGCT
and
TTTGCTGTAGAGGATGCAAT
(for D7S478);
TTCGGGCTCTCTGTTATAAA
and
CCGAAGCAGGATTTTATTTC
(for D7S670);
AGCTGCCAGGAATCAACTGAGAG
and
GATGCTCACATAAAGGAGGGAGG
(for D8S258);
CCAATACCTGCAGTAGTGCC
and
GAGCTGCTTAACACATAGGG
(for NEFL);
CACCACAGACATCTCACAACC
and
CCAGTGAATAGTTCAGGGATGG
(for D10S541);
AGGGTTATGTATAACCGACTCC
and
GTCTAAGCCCTCGAGTTGTGG
(for D13S153);
GGTTCACAATTGGACAGTAT
and
GAACCCTCCATGCTGACATT
(for D16S400);
GTACCCATGTACCCCCAATA
and
CAAAGCACCACATAGACTAA
(for D16S402);
GAGAGGAAGGTGGAAATACA
and
GTTTAGCAGAATGAGAATAT
(for D16S422);
AATAAATTCCCACTGCCACTC
and
ATCCCCTGAGGGATACTATTC
(for p53);
GGATGGCCTTTTAGAAAGTGG
and
ACACAGACTTGTCCTACTGCC
(for D17S855).

9. Method according to claim 5, wherein the reproduced DNA fragments are split and analysed by capillary electrophoresis.

10. Method according to claim 1, wherein the isolation or concentration of tumour cells cytokeratin-positive cells were isolated from sample material, and/or positive epithelial cells for tissue specific proteins.

11. Method according to claim 10, wherein epithelial cells are concentrated from sample material by means of density gradient centrifugation-if necessary after homogenisation in a solvent, and cytokeratin-positive and/or positive cell clusters from tissue specific proteins are then split off by means of immunomagnetic cell isolation.

12. Method according to claim 11, wherein the medium for the density gradient centrifugation is a hyper-osmotic medium.

13. Method according to claim 12, wherein the hyper-osmotic buffer consists of one of the following mediums: 13.8% (w/v) Diatrizoate and 8% (w/v) dextran 500 in H₂O (polymorphprep) or 13% (w/v) Nycodenz, 0.58% (w/v) NaCl and 5 mM Tricine-NaOH pH 7.4 in H₂O (Nycoprep).

14. Method according to claim 1, wherein genetic changes in the isolated cell clusters are analysed by means of cluster analysis.

15. Application of a method according to claim 1 for the molecular characterization of tumours or tumour sections or for the determination of clonality from cells clusters isolated from sample material as well as for the detection of a tumour to determine the tumour stage, the metastasising potential, therapy requirements, efficacy of therapy of a tumour or part thereof, as well as the assessment of the course of a disease or therapy.

16. Application according to claim 15 for the detection and/or characterisation of tumours or tumour areas of the following carcinomas: mamma-, ovarial-, colon-, gastric-, prostate and/or bladder carcinoma.


SEQUENCE LISTING (42 sequences)

SEQ ID NO: 1 - DNA, 20 bp, Artificial Sequence
  Description of the artificial Sequence Oligonucleotide
gcaggacatgagatgactga

SEQ ID NO: 2 - DNA, 20 bp, Artificial Sequence
  Description of the artificial Sequence Oligonucleotide
gttatgccactccctcacac

SEQ ID NO: 3 - DNA, 22 bp, Artificial Sequence
  Description of the artificial Sequence Oligonucleotide
gtttgaagaatttgagccaacc

SEQ ID NO: 4 - DNA, 20 bp, Artificial Sequence
  Description of the artificial Sequence Oligonucleotide
ttcttctgcacacttggcac

SEQ ID NO: 5 - DNA, 22 bp, Artificial Sequence
  Description of the artificial Sequence Oligonucleotide
ctcgaggtctcatcctctttcc

SEQ ID NO: 6 - DNA, 22 bp, Artificial Sequence
  Description of the artificial Sequence Oligonucleotide
gcagaggtgcacaaaggagtaa

SEQ ID NO: 7 - DNA, 22 bp, Artificial Sequence
  Description of the artificial Sequence Oligonucleotide
aggcccacagaggagataacag

SEQ ID NO: 8 - DNA, 22 bp, Artificial Sequence
  Description of the artificial Sequence Oligonucleotide
caggtgtggtagatgccaaaga

SEQ ID NO: 9 - DNA, 22 bp, Artificial Sequence
  Description of the artificial Sequence Oligonucleotide
gcaacttatccaaaccctgacc

SEQ ID NO: 10 - DNA, 25 bp, Artificial Sequence
  Description of the artificial Sequence Oligonucleotide
agagtggactaggaaatgctaggag

SEQ ID NO: 11 - DNA, 22 bp, Artificial Sequence
  Description of the artificial Sequence Oligonucleotide
agttcctgactgggaattcgat

SEQ ID NO: 12 - DNA, 22 bp, Artificial Sequence
  Description of the artificial Sequence Oligonucleotide
ttggccaaattacacacctttg

SEQ ID NO: 13 - DNA, 17 bp, Artificial Sequence
  Description of the artificial Sequence Oligonucleotide
ttccatttgtctcggtt

SEQ ID NO: 14 - DNA, 20 bp, Artificial Sequence
  Description of the artificial Sequence Oligonucleotide
agtctcctcgtctcacacct

SEQ ID NO: 15 - DNA, 20 bp, Artificial Sequence
  Description of the artificial Sequence Oligonucleotide
cagtgctggagttgttcaag

SEQ ID NO: 16 - DNA, 20 bp, Artificial Sequence
  Description of the artificial Sequence Oligonucleotide
ctgggagtcaagtgttttgg

SEQ ID NO: 17 - DNA, 20 bp, Artificial Sequence
  Description of the artificial Sequence Oligonucleotide
tgctaagtcttgattttgcc

SEQ ID NO: 18 - DNA, 18 bp, Artificial Sequence
  Description of the artificial Sequence Oligonucleotide
aacggtcatctgtgttcg

SEQ ID NO: 19 - DNA, 20 bp, Artificial Sequence
  Description of the artificial Sequence Oligonucleotide
ggtgtttgtgtcattacgct

SEQ ID NO: 20 - DNA, 20 bp, Artificial Sequence
  Description of the artificial Sequence Oligonucleotide
tttgctgtagaggatgcaat

SEQ ID NO: 21 - DNA, 20 bp, Artificial Sequence
  Description of the artificial Sequence Oligonucleotide
ttcgggctctctgttataaa

SEQ ID NO: 22 - DNA, 20 bp, Artificial Sequence
  Description of the artificial Sequence Oligonucleotide
ccgaagcaggattttatttc

SEQ ID NO: 23 - DNA, 23 bp, Artificial Sequence
  Description of the artificial Sequence Oligonucleotide
agctgccaggaatcaactgagag

SEQ ID NO: 24 - DNA, 23 bp, Artificial Sequence
  Description of the artificial Sequence Oligonucleotide
gatgctcacataaaggagggagg

SEQ ID NO: 25 - DNA, 20 bp, Artificial Sequence
  Description of the artificial Sequence Oligonucleotide
ccaatacctgcagtagtgcc

SEQ ID NO: 26 - DNA, 20 bp, Artificial Sequence
  Description of the artificial Sequence Oligonucleotide
gagctgcttaacacataggg

SEQ ID NO: 27 - DNA, 21 bp, Artificial Sequence
  Description of the artificial Sequence Oligonucleotide
caccacagacatctcacaacc

SEQ ID NO: 28 - DNA, 22 bp, Artificial Sequence
  Description of the artificial Sequence Oligonucleotide
ccagtgaatagttcagggatgg

SEQ ID NO: 29 - DNA, 22 bp, Artificial Sequence
  Description of the artificial Sequence Oligonucleotide
agggttatgtataaccgactcc

SEQ ID NO: 30 - DNA, 21 bp, Artificial Sequence
  Description of the artficial Sequence Oligonucleotide
gtctaagccctcgagttgtgg

SEQ ID NO: 31 - DNA, 20 bp, Artificial Sequence
  Description of the artificial Sequence Oligonucleotide
ggttcacaattggacagtat

SEQ ID NO: 32 - DNA, 20 bp, Artificial Sequence
  Description of the artificial Sequence Oligonucleotide
gaaccctccatgctgacatt

SEQ ID NO: 33 - DNA, 20 bp, Artificial Sequence
  Description of the artificial Sequence Oligonucleotide
gtacccatgtacccccaata

SEQ ID NO: 34 - DNA, 20 bp, Artificial Sequence
  Description of the artificial Sequence Oligonucleotide
caaagcaccacatagactaa

SEQ ID NO: 35 - DNA, 20 bp, Artificial Sequence
  Description of the artificial Sequence Oligonucleotide
gagaggaaggtggaaataca

SEQ ID NO: 36 - DNA, 20 bp, Artificial Sequence
  Description of the artificial Sequence Oligonucleotide
gtttagcagaatgagaatat

SEQ ID NO: 37 - DNA, 21 bp, Artificial Sequence
  Description of the artificial Sequence Oligonucleotide
aagaaattcccactgccactc

SEQ ID NO: 38 - DNA, 21 bp, Artificial Sequence
  Description of the artificial Sequence Oligonucleotide
atcccctgagggatactattc

SEQ ID NO: 39 - DNA, 21 bp, Artificial Sequence
  Description of the artificial Sequence Oligonucleotide
ggatggccttttagaaagtgg

SEQ ID NO: 40 - DNA, 21 bp, Artificial Sequence
  Description of the artificial Sequence Oligonucleotide
acacagacttgtcctactgcc

SEQ ID NO: 41 - DNA, 21 bp, Artificial Sequence
  Description of the artificial Sequence Oligonucleotide
ttcaccctcagaaggtgacca

SEQ ID NO: 42 - DNA, 23 bp, Artificial Sequence
  Description of the artificial Sequence Oligonucleotide
ccagcgtccagcacacagcatga